(19) United States
(12) Patent Application Publication
(10) Pub. No.: US 2024/0342775 A1
(43) Pub. Date: Oct. 17, 2024

(54) FOOD WASTE DISPOSER AND CONTROL METHOD THEREFOR

(71) Applicant: SAMSUNG ELECTRONICS CO., LTD., Suwon-si Gyeonggi-do (KR)

(72) Inventors: Hyunnam LEE, Suwon-si (KR); Namsoo PARK, Suwon-si (KR); Keonpyo KOO, Suwon-si (KR); Kyoungmok KIM, Suwon-si (KR); Daegeon KIM, Suwon-si (KR); Jungkwon KIM, Suwon-si (KR); Wonyoung SEO, Suwon-si (KR); Yongjun HWANG, Suwon-si (KR)

(73) Assignee: SAMSUNG ELECTRONICS CO., LTD., Suwon-si Gyeonggi-do (KR)

(21) Appl. No.: 18/756,079

(22) Filed: Jun. 27, 2024

Related U.S. Application Data
(63) Continuation of application No. PCT/KR2022/017458, filed on Nov. 8, 2022.

(30) Foreign Application Priority Data
Dec. 29, 2021 (KR) 10-2021-0190914

(51) Int. Cl. B09B 3/35; A61L 9/014; A61L 9/20; B02C 23/26; B09B 3/38; B09B 3/40; B09B 101/70

(52) U.S. Cl. CPC B09B 3/35; A61L 9/014; A61L 9/205; B02C 23/26; B09B 3/38; B09B 3/40; A61L 2209/11; A61L 2209/12; A61L 2209/22; B09B 2101/70

(57) ABSTRACT

A food waste disposer comprising a chamber for accommodating food for disposal; a deodorization device for deodorizing gas and discharging same to the outside; and a cover member for covering the chamber. The cover member includes a fan, which is arranged to face the inside of the chamber so as to generate, when being driven, a convection current inside the chamber; and a discharge pipe arranged such that a discharge port formed at a first end portion is arranged on outside a path of upward flow of air such that the upward flow of air bypasses the fan, and a second end portion, which is opposite to the first end portion, is connected to the deodorization device.

### CROSS-REFERENCE TO RELATED APPLICATION

This application is a continuation application, under 35 U.S.C. § 111(a), of international application No. PCT/KR2022/017458, filed on Nov. 8, 2022, which claims priority under 35 U. S. C. § 119 to Korean Patent Application No. 10-2021-0190914, filed on Dec. 29, 2021, the disclosures of which are incorporated herein by reference in their entireties.

### BACKGROUND

### Field

Various embodiments of the disclosure relate to a food waste disposer and a method for controlling the same.

### Description of the Related Art

A food waste disposer is a device for treating food waste by grinding/stirring, heating, and drying food waste contained in a chamber. The food waste disposer may include a fan for quickly drying food waste and convecting air inside the chamber. The gas generated by heating the food waste may be moved to a deodorizer along the fan. In this case, the crushed food waste may be blown away by the fan to be stuck in a portion of the fan, deteriorating the performance of the fan.

Further, the time required to treat the food waste may vary according to characteristics such as the amount of food waste in the chamber, humidity, or strength. Therefore, if the operation time of the food waste treatment is constant, the operation time may be longer than the time required for the food waste to be treated, and thus power may be wasted, or the operation time may be less than the time required for the food waste to be treated, causing moisture to remain in the food waste.

### SUMMARY

Various embodiments of the disclosure may provide a discharge pipe structure to provide a path along which the water vapor in the chamber bypasses the fan.

Various embodiments of the disclosure may provide a controller for controlling the operation of the food waste disposer using the temperature in the chamber, humidity, or the torque of the stirring motor.

A food waste disposer according to an embodiment of the disclosure may comprise a chamber disposed to receive food waste to be treated, a deodorizer disposed to deodorize gas and discharge the gas to an outside, and a cover member arrangeable to cover the chamber. The cover member may include a fan arrangeable to face inward of the chamber while the cover member is arranged to cover the chamber so that while the fan is arranged to face inward of the chamber, the fan generates convection in the chamber while being driven, and a discharge pipe including a discharge port formed at a first end portion arrangeable outside a path of upward flow of air such that the upward flow of air bypasses the fan, and a second end portion opposite to the first end portion connectable to the deodorizer.

A food waste disposer performing a process of stirring, grinding, or heating food waste, according to an embodiment of the disclosure, may comprise a chamber to accommodate food waste, a stirrer disposed in the chamber and provided to stir and crush the food waste, a driving device arrangeable to rotate the stirrer, a temperature detector to detect a temperature in the chamber, a humidity detector treating device to detect a humidity in the chamber, a heating device arrangeable outside the chamber, a cover member including a fan disposed to face inward of the chamber and a fan driver disposed to rotate the fan, and provided to cover the chamber, and a controller provided to control an overall operation of the driving device, the heating device, or the fan driver. The controller may determine an end time of the treating process using humidity in the chamber and current flowing through the driving device.

A method for controlling a food waste disposer, according to an embodiment of the disclosure, may comprise treating food waste in a chamber by stirring, crushing, or heating to dry, detecting a humidity in the chamber or a magnitude of a current flowing through a driving motor driven to stir the food waste, and determining whether to terminate treating the food waste according to the detected humidity and the magnitude of the current flowing through the driving motor.

According to various embodiments proposed in the disclosure, as the water vapor in the chamber is discharged while bypassing the fan, it is possible to prevent food waste from getting stuck in the fan.

According to various embodiments proposed in the disclosure, the controller may control the operation of the fan using the temperature in the chamber, humidity, or the torque of the stirring motor, and determine the end time of food waste treatment, shortening the food waste treatment time.

### BRIEF DESCRIPTION OF DRAWINGS

FIG. 1 is a perspective view illustrating a food waste disposer according to an embodiment of the disclosure;

FIG. 2 is a perspective view illustrating a state in which a cover member of the food waste disposer of FIG. 1 is open according to an embodiment of the disclosure;

FIG. 3 is a rear perspective view of the food waste disposer of FIG. 1 according to an embodiment of the disclosure;

FIG. 4 is a cross-sectional view of the food waste disposer of FIG. 1 according to an embodiment of the disclosure;

FIG. 5 is a perspective view illustrating a state in which a housing and a cover member are removed from a food waste disposer according to an embodiment of the disclosure;

FIG. 6 is an exploded perspective view of the food waste disposer of FIG. 5 according to an embodiment of the disclosure;

FIG. 7 is an exploded perspective view illustrating a cover member of a food waste disposer according to an embodiment of the disclosure;

FIG. 8A is a bottom view illustrating a cover member of a food waste disposer according to an embodiment of the disclosure;

FIG. 8B is a bottom view with a grill removed from FIG. 8A according to an embodiment of the disclosure;

FIG. 9 is a view illustrating a discharge pipe and an end cap of a cover member according to an embodiment of the disclosure;

FIG. 10 is a rear view illustrating a cover member of a food waste disposer according to an embodiment of the disclosure;

FIG. 11A is a cross-sectional view illustrating a state in which a cover member according to an embodiment of the disclosure is open;

FIG. 11B is a cross-sectional view illustrating a state in which a cover member according to an embodiment is closed;

FIG. 12 is a block view illustrating a food waste disposer according to an embodiment of the disclosure;

FIG. 13 is a flowchart illustrating a method for controlling a food waste disposer according to an embodiment of the disclosure;

FIG. 14 is a flowchart illustrating a method for controlling an operation of a fan driver for rotating a convective fan of FIG. 13 according to an embodiment of the disclosure;

FIG. 15 is a graph illustrating an operation of a fan driver during an operation of a food waste disposer according to an embodiment of the disclosure; and

FIG. 16 is a flowchart illustrating a method for determining an end time of a food waste treating by a food waste disposer according to an embodiment of the disclosure.

### DETAILED DESCRIPTION

Embodiments of the present invention are now described with reference to the accompanying drawings in such a detailed manner as to be easily practiced by one of ordinary skill in the art. However, the disclosure may be implemented in other various forms and is not limited to the embodiments set forth herein. The same or similar reference denotations may be used to refer to the same or similar elements throughout the specification and the drawings. Further, for clarity and brevity, no description is made of well-known functions and configurations in the drawings and relevant descriptions.

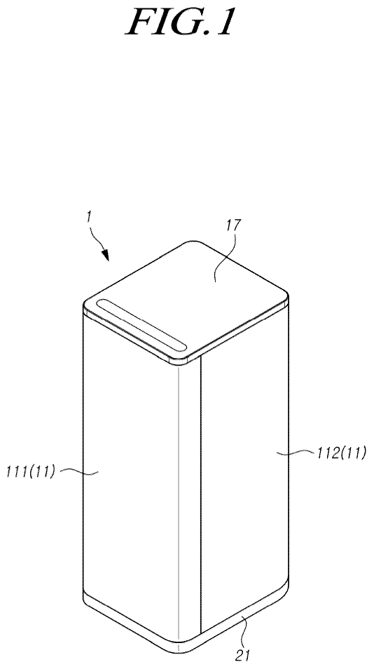

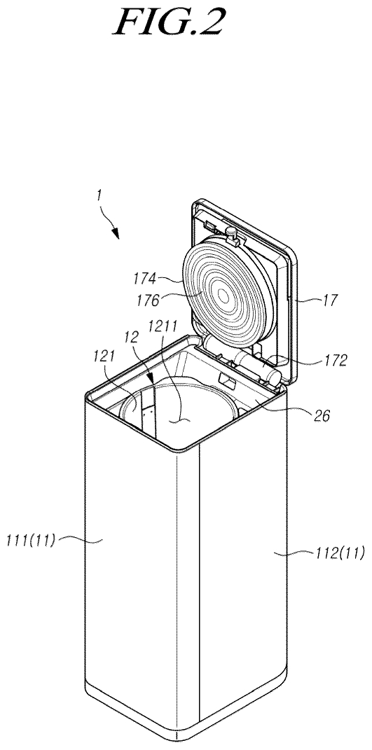

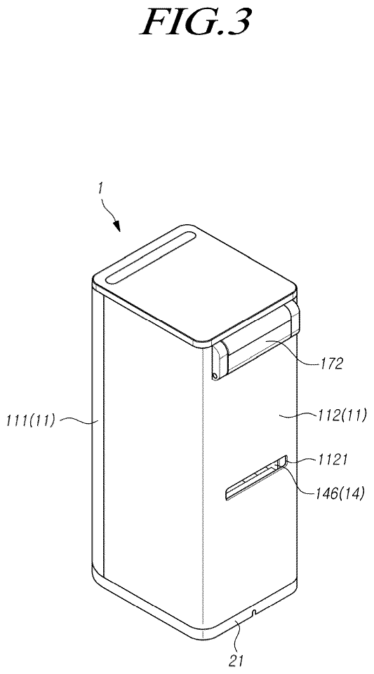

FIG. 1 is a perspective view illustrating a food waste disposer according to an embodiment of the disclosure. FIG. 2 is a perspective view illustrating a state in which a cover member of the food waste disposer of FIG. 1 is open. FIG. 3 is a rear perspective view of the food waste disposer of FIG. 1.

Referring to FIGS. 1 to 3, a food waste disposer 1 according to an embodiment of the disclosure may include a housing 11, a cover member 17, or a treating device 12.

According to an embodiment, the housing 11 may include a front housing 111 or a rear housing 112. The housing 11 may form, e.g., an outer appearance of the food waste disposer 1. The housing 11 may be disposed, e.g., on a lower frame 21 forming the bottom surface of the food waste disposer 1.

The front housing 111 may form, e.g., a front exterior of the food waste disposer 1. The rear housing 112 may form, e.g., a rear exterior of the food waste disposer 1. The front housing 111 may be provided to be detachable from, e.g., the rear housing 112. The user may access various components disposed inside the food waste disposer 1 by separating the front housing 111 or the rear housing 112.

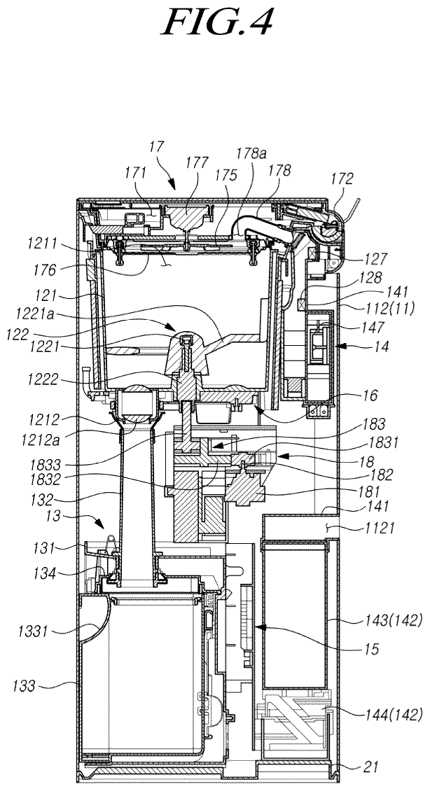

According to an embodiment, the rear housing 112 may include an exhaust hole 1121. The exhaust hole 1121 may be formed to communicate with, e.g., an outlet 146 of a deodorizer (e.g., the deodorizer 14 of FIG. 4). The deodorizer (e.g., the deodorizer 14 of FIG. 4) may be a device for filtering air including the odor generated in the chamber 121 of the treating device 12. The air filtered by the deodorizer (e.g., the deodorizer 14 of FIG. 4) may be discharged to the outside through the exhaust hole 1121.

According to an embodiment, the cover member 17 may include a hinge 172, a treating device cover 174, a fan 175, or a grill 176. The cover member 17 may be provided to be rotatable with respect to the housing 11 through a hinge 172 connected to the upper frame 26. The treating device cover 174 may be provided to open and close the chamber 121, and may convect air in the chamber 121 by rotating the fan 175 in a state in which the chamber 121 is closed. Details of the cover member 17 are described below.

According to an embodiment, the treating device 12 may include a chamber 121 and a stirrer (e.g., the stirrer 122 of FIG. 4). The user may open the cover member 17 to contain food waste to be treated through an opening 1211 of the chamber 121, and then close the cover member 17 again. Food received in the chamber 121 may be crushed or stirred by a stirrer (e.g., the stirrer 122 of FIG. 4). Details of the treating device 12 are described below.

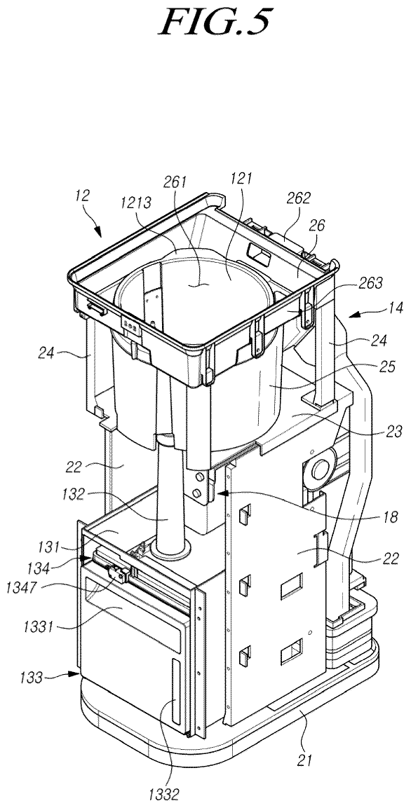

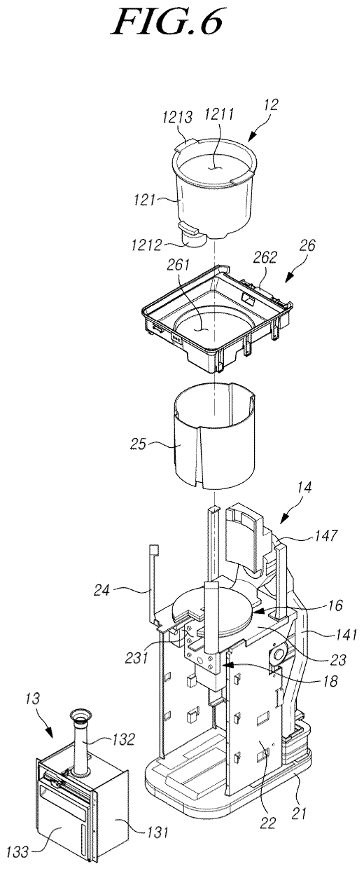

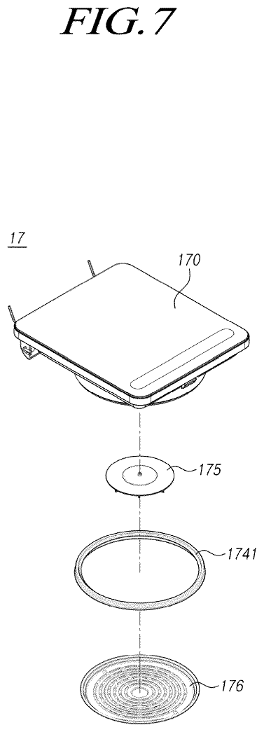

FIG. 4 is a cross-sectional view of the food waste disposer of FIG. 1. FIG. 5 is a perspective view illustrating a state in which a housing and a cover member are removed from a food waste disposer according to an embodiment of the disclosure. FIG. 6 is an exploded perspective view of the food waste disposer of FIG. 5. FIG. 7 is an exploded perspective view illustrating a cover member of a food waste disposer according to an embodiment of the disclosure.

Referring to FIGS. 4 to 7, a food waste disposer 1 according to an embodiment of the disclosure may include a housing 11, a treating device 12, a collecting device 13, a deodorizer 14, a heating device 16, a cover member 17, or a controller 15. The housing 11, the cover member 17, or the treating device 12 illustrated may be identical in whole or part to the housing 11, the cover member 17, or the treating device 12 illustrated in FIGS. 1 to 3.

According to an embodiment, the housing 11 may form an accommodating space in which the components of the food waste disposer 1 are disposed. The housing 11 may be provided to protect, e.g., components disposed in the accommodating space from external shocks.

According to various embodiments, the treating device 12 may include a chamber 121, a stirrer 122, or a driving device 18. The treating device 12 may be, e.g., a device for accommodating food waste and grinding, stirring, or heating to dry the food waste.

According to an embodiment, an upper portion of the chamber 121 may be open to form an opening 1211. The opening 1211 may be opened and closed by the cover member 17. The opening 1211 may be, e.g., cylindrical, but is not limited thereto. The chamber 121 may form a space in which food waste may be crushed, stirred, or heated. Hereinafter, grinding, stirring, or heating to dry food waste is defined as treating the food waste. Food waste may be introduced from an upper portion of the chamber 121.

According to an embodiment, an opening/closing part 1212 may be disposed at a portion of a lower portion of the chamber 121. The opening/closing part 1212 may be provided, e.g., to treat food waste and discharge remaining by-products from the chamber 121. The opening/closing part 1212 may be closed while the food waste is being treated, and may be opened after the food waste treatment is completed. The opening/closing part 1212 may be opened or closed by, e.g., a ball valve 1212a disposed therein, but is not limited thereto. The opening/closing part 1212 may be closed when the food waste disposer 1 does not operate. The by-product may pass through the opening/closing part 1212 and be transferred to a storage container 133 storing the by-product.

According to an embodiment, a handle portion 1213 formed to partially protrude radially outward may be disposed at an upper end of the chamber 121. The user may detach the chamber 121 from the food waste disposer 1 using the handle portion 1213.

According to an embodiment, the stirrer 122 may include a rotation grinder 1221 or a power transmission member 1222. One end of the power transmission member 1222 may be connected to the rotating grinder 1221, and the other end of the power transmission member 1222 may be connected to the driving device 18. The rotating grinder 1221 may be connected to, e.g., the power transmission member 1222 to rotate about the power transmission member 1222. The rotating grinder 1221 may include, e.g., a plurality of blades 1221a. Each of the plurality of blades 1221a may be disposed to have a different height. The treating device 12 may grind or stir food waste filling the chamber 121 by rotating, e.g., the rotating grinder 1221. In other words, the stirrer 122 may grind and stir the food waste in the chamber 121 so that the food waste is evenly heated.

According to an embodiment, the driving device 18 may include a driving motor 181, a shaft 182, or a gear assembly 183. For example, one side of the shaft 182 may be connected to the driving motor 181 and the other side of the shaft 182 may be connected to the gear assembly 183. Specifically, the other side of the shaft 182 may be connected to a first gear 1831 of the gear assembly 183. The shaft 182 may transfer, e.g., the rotational power of the driving motor 181 to the gear assembly 183. The rotational power transferred to the gear assembly 183 may be transferred to the power transmission member 1222.

According to an embodiment, the gear assembly 183 may include a first gear 1831, a second gear 1832, or a third gear 1833, but the number of gears constituting the gear assembly 183 is not limited thereto. The first gear 1831 may be coupled to, e.g., the shaft 182 and may be disposed to rotate with driving of the driving motor 181. The second gear 1832 may be rotatably disposed to engage with, e.g., the first gear 1831. The third gear 1833 may be rotatably disposed to engage with, e.g., the second gear 1832. The third gear 1833 may be coupled to, e.g., the power transmission member 1222 to transfer the rotational power transferred from the driving motor 181 to the power transmission member 1222.

According to an embodiment, the driving device 18 may transfer rotational power to the power transmission member 1222. The power transmission member 1222 may be disposed to penetrate, e.g., a portion of a lower surface of the chamber 121. For example, the power transmission member 1222 may be disposed so that a portion thereof is positioned inside the chamber 121 and another portion is positioned outside the chamber 121. In other words, among the components connected to two opposite ends of the power transmission member 1222, the rotating grinder 1221 may be positioned inside the chamber 121, and the driving device 18 may be positioned outside the chamber 121. The stirrer 122 may be disposed, e.g., under the chamber 121.

According to an embodiment, the heating device 16 may be disposed adjacent to an outer surface of the chamber 121. The heating device 16 may be positioned, e.g., under the chamber 121, but is not limited thereto. The heating device 16 may generate heat, e.g., by heating wires provided therein. When the temperature inside the chamber 121 is increased by the heating device 16, moisture in the food waste may be evaporated and dried. The evaporated water vapor is discharged to the deodorizer 14 to be described below and, after the odor or the like is filtered, is discharged to the outside.

The heating device 16 may be disposed, e.g., between the chamber 121 and an accommodating frame 25 provided to fix the position of the chamber 121. Here, the accommodating frame 25 may be a frame that covers the outside of the treating device 12 or the heating device 16 so that the treating device 12 or the heating device 16 is more stably supported and fixed inside the food waste disposer 1.

According to an embodiment, the cover member 17 may include a hinge 172, a treating device cover 174, a fan 175, a grill 176, a fan driver 177, a discharge pipe 178, or an end cap 179.

According to an embodiment, the treating device cover 174 may be disposed under the cover member 17 to open and close the opening 1211 above the chamber 121. The treating device cover 174 may have, e.g., a shape corresponding to the shape of the upper surface of the chamber 121. When the treating device cover 174 closes the upper surface of the chamber 121, the air inside the chamber 121 may pass through the discharge pipe 178 of the cover member 17 and flow to the deodorizer 14. An outer circumferential surface of the treating device cover 174 may be surrounded by a packing portion 1741.

According to an embodiment, the hinge 172 may be disposed at a portion adjacent to an upper end of the chamber 121. The hinge 172 may be positioned, e.g., at a position corresponding to a hinge mounting portion 262 of the upper frame 26 to be described below. The hinge 172 may be mounted on, e.g., the hinge mounting portion 262 of the upper frame 26. Accordingly, the cover member 17 may be opened and closed by rotating about the hinge 172. The user may open the cover member 17 to put food waste into the chamber 121.

According to an embodiment, the cover member 17 may be provided with an inner space 171 so that components may be disposed therein. For example, the fan 175, the fan driver 177, or the discharge pipe 178 may be disposed in the inner space 171. The inner space 171 of the cover member 17 may be formed by, e.g., the cover member housing 170. In other words, the fan 175, the fan driver 177, or the discharge pipe 178 may be disposed inside the cover member housing 170.

A portion of the lower portion of the cover member 17 may be formed to be open. Specifically, the treating device cover 174 of the cover member 17 may be formed to have an open lower portion. Accordingly, the lower portion of the treating device cover 174 may be open to allow the flow of air generated by the rotation of the fan 175 to be transferred into the chamber 121, forming convection. For example, a grill 176 may be disposed in the open portion of the lower portion of the cover member 17.

According to an embodiment, the fan 175 may be disposed in the inner space 171 of the cover member 17. The fan 175 may be disposed to rotate about, e.g., a portion of the cover member 17 adjacent to the center of the treating device cover 174. The food waste disposer 1 may convect air in the chamber 121 by rotating, e.g., the fan 175. In other words, when the chamber 121 is heated, the food waste disposer 1 may rotate the fan 175 to circulate the air in the chamber 121, thereby forming a uniform internal temperature. When the convective fan 175 rotates, a low pressure may be formed in a central portion of the chamber 121, and a high pressure may be formed in a portion other than the center. Accordingly, an upward airflow may be generated in the central portion of the chamber 121.

According to an embodiment, the fan driver 177 may be disposed in the inner space 171 of the cover member 17 to rotate the convective fan 175. The fan driver 177 may be controlled by, e.g., the controller 15 according to an environment inside the chamber 121 or torque applied to the driving device 18 that rotates the stirrer 122 while the food waste is being treated. Here, the environment inside the chamber 121 may be humidity or temperature, but is not limited thereto.

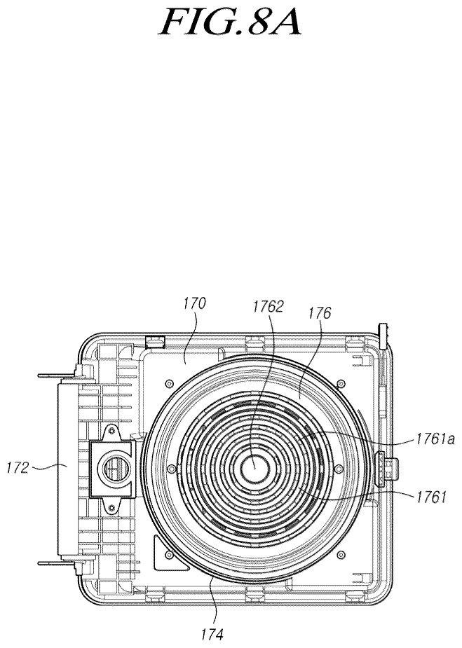

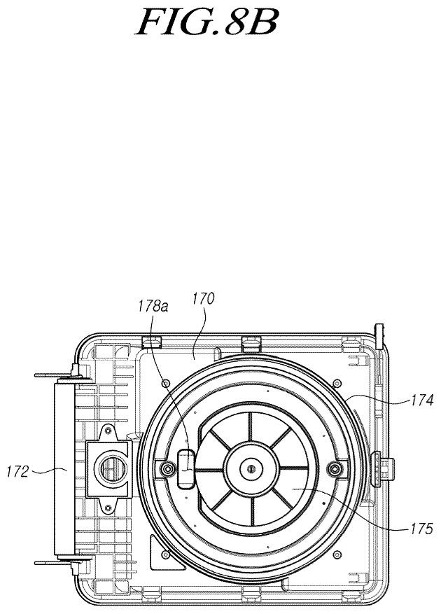

According to an embodiment, the grill 176 may be disposed under the fan 175. The grill 176 may be disposed, e.g., on a lower surface of the treating device cover 174. In other words, the grill 176 may be disposed between the chamber 121 and the fan 175. The grill 176 may be provided with a plurality of holes (e.g., the plurality of holes 1761a of FIG. 8A), and the flow of air generated by the rotation of the fan 175 may be transferred to the chamber 121 through the plurality of holes (e.g., the plurality of holes 1761a of FIG. 8A). The grill 176 may be fixed to the cover member 17, e.g., by screw coupling.

According to an embodiment, the discharge pipe 178 may be provided so that the discharge port 178a is positioned outside the convective fan 175. The discharge port 178a may be positioned at a first end portion 1781 connected to the chamber 121 in the discharge pipe 178. The discharge port 178a may be positioned above the fan 175, outside the fan 175. The discharge pipe 178 may be disposed so that the opposite end of the discharge port 178a is connected to the exhaust pipe 141 of the deodorizer 14. In other words, the discharge pipe 178 is a component for connecting the chamber 121 and the exhaust pipe 141 of the deodorizer 14, and may be provided to allow the gas in the chamber 121 to flow to the deodorizer 14.

According to an embodiment, the end cap 179 may be disposed at a second end 1782 which is an opposite end of the first end portion 1781 of the discharge pipe 178.

Although not shown, a locking device (not shown) may be disposed in the cover member 17 or the chamber 121. The locking device may be provided to lock the cover member 17 so that the user may not open the inside while treating the food waste.

According to various embodiments, the collecting device 13 may include a storage container 133, a storage container cover 134, a case 131, or a transfer pipe 132. The collecting device 13 is, e.g., a device for accommodating and storing by-products treated by grinding, stirring, or heating of food waste in the treating device 12 from the chamber 121. The collecting device 13 may have a structure sealed so that stored by-products or odors that may be generated from the by-products do not leak to the outside.

The storage container 133 may include, e.g., a grip portion 1331 or a transparent window 1332. The user may mount or detach the storage container 133 from the case 131 using the grip portion 1331. The transparent window 1332 may be disposed to be visible from the outside when the storage container 133 is mounted on the case 131. The transparent window 1332 may be formed along the height direction of the storage container 133 so that the user may observe the height of the by-product inside. The grip portion 1331 or the transparent window 1332 may be positioned on a surface corresponding to the open surface of the case 131 when the storage container 133 is mounted on the case 131.

The transfer pipe 132 may be, e.g., a component connecting the chamber 121 and the storage container 133. By-products generated by being treated in the chamber 121 may be transferred to the storage container 133 along the transfer pipe 132. The by-products are transferred from the chamber 121 to the storage container 133 in a free-fall manner.

The case 131 may be disposed to be surrounded by, e.g., a lower frame 21 or a side frame 22 to be described below. The case 131 may be provided with an inner space in which the storage container 133 is mounted. The user may detach the storage container 133 from the case 131 or mount the storage container 133 to the case 131.

The storage container cover 134 may be provided to seal the storage container 133 when the storage container 133 is mounted on the case 131. The storage container cover 134 may be disposed to be fixed to an upper portion in the inner space of the case 131.

According to various embodiments, the deodorizer 14 may include an exhaust pipe 141, a deodorizing part 142, an outlet 146, or a deodorizing fan 147. The deodorizer 14 is a device that filters and then discharges the odor when the gas containing the odor generated from the food waste received in the chamber 121 is discharged to the outside of the food waste disposer 1.

According to an embodiment, the deodorizing part 142 may include an activated carbon filter 143 or an optical filter module 144. The gas passing through the deodorizing part 142 may be primarily photolyzed and filtered by the optical filter module 144, and then may be secondarily filtered while passing through the activated carbon filter 143. Before the gas introduced from the chamber 121 is filtered by the activated carbon filter 143, the gas may be primarily filtered by the optical filter module 144 to extend the service life of the activated carbon filter 143. The gas filtered by the deodorizing part 142 may be discharged to the discharge port 146. The outlet 146 may be connected to the exhaust hole 1121 of the rear housing 112, and the filtered gas may be discharged to the exhaust hole 1121.

According to an embodiment, a photocatalytic filter may be provided in the optical filter module 144. The photocatalytic filter may be formed of, e.g., titanium dioxide. For example, when light energy in an ultraviolet band is applied to the photocatalytic filter, the strong reducing power of hydrogen peroxide and the strong oxidizing power of the hydroxyl group are generated on the surface thereof, thereby decomposing various contaminants and harmful components in the gas.

For example, activated carbon having a size of 1 mm to 10 mm may be used in the activated carbon filter 143. The activated carbon may deodorize, e.g., the odor of the gas passing through the optical filter module 144.

According to an embodiment, the deodorizing fan 147 may be disposed in the exhaust pipe 141. The deodorizing fan 147 may be dosposed, e.g., on a flow path in the deodorizer 14 to flow gas passing through the inside of the deodorizer 14.

According to an embodiment, the food waste disposer 1 may further include a temperature detector 127 or a humidity detector 128. The temperature detector 127 or the humidity detector 128 may be provided to directly or indirectly detect, e.g., temperature or humidity in the chamber 121. FIG. 4 schematically illustrates the temperature detector 127 and the humidity detector 128.

According to an embodiment, the temperature detector 127 may be disposed in the deodorizer 14. The temperature detector 127 may be disposed, e.g., in the exhaust pipe 141 of the deodorizer 14. Specifically, as illustrated, the temperature detector 127 may be disposed adjacent to the discharge pipe 178 in the exhaust pipe 141 to detect the temperature of the gas immediately after the gas in the chamber 121 passes through the discharge pipe 178 and flows into the exhaust pipe 141. The temperature detector 127 may indirectly detect the temperature in the chamber 121 by detecting the temperature of the gas passing through the discharge pipe 178.

According to an embodiment, the temperature detector 127 may be disposed in the treating device 12. In particular, the temperature detector 127 may be disposed on an upper portion of the chamber 121 to directly detect the temperature in the chamber 121.

According to an embodiment, the humidity detector 128 may be disposed in the deodorizer 14. The humidity detector 128 may be disposed, e.g., in the exhaust pipe 141 of the deodorizer 14. Specifically, the humidity detector 128 may be disposed above the deodorizing fan 147 as illustrated. The humidity detector 128 may indirectly detect the humidity in the chamber 121 by detecting the humidity of the gas in the exhaust pipe 141. According to an embodiment, the humidity detector 128 may be disposed adjacent to the temperature detector 127.

According to an embodiment, the humidity detector 128 may be disposed in the treating device 12. In particular, the humidity detector 127 may be disposed above the chamber 121 to directly detect humidity in the chamber 121.

The temperature detector 127 or the humidity detector 128 may transmit, e.g., information about temperature or humidity to the controller 15. The controller 15 may control operations of devices used to treat food waste, such as the stirrer 122, the fan 175, the fan driver 177, the driving device 18, or the heating device 16, using the received information about temperature or humidity.

According to an embodiment, the controller 15 may control the overall operation of the electronic components of the food waste disposer 1. In other words, the controller 15 may perform overall control necessary for treating the food waste put into the chamber 121.

For example, the controller 15 may perform control to rotate the stirrer 122 by operating the driving device 18 to crush or stir food waste. For example, the controller 15 may perform control to heat the heating wire in the heating device 16 to heat the inside of the chamber 121. For example, the controller 15 may control the operation of the fan 175 to convect the gas in the chamber 121. For example, the controller 15 may perform control to open the opening/closing part 1212 to transfer the remaining by-products to the storage container 133 after the food waste is treated.

The controller 15 may adjust the heat generation intensity of the heating device 16 or the rotation intensity of the fan 175 using, e.g., the information about the temperature or humidity in the chamber 121 received from the temperature detector 127 or the humidity detector 128, thereby enhancing the operation efficiency of the food waste disposer 1. Further, the controller 15 may determine whether the storage container 133 is filled with a specific amount or more by-products using a storage amount detector (e.g., the storage amount detector 1341 of FIG. 6) and perform control to visually or audibly notify the user.

The food waste disposer 1 may include a plurality of frames 20 for supporting or fixing components in the housing 11. According to various embodiments, the plurality of frames 20 may include a lower frame 21, a side frame 22, an intermediate frame 23, a supporting frame 24, an accommodating frame 25, or an upper frame 26.

According to an embodiment, the lower frame 21 may form a bottom surface of the food waste disposer 1. The lower frame 21 may support, e.g., the front housing 111 or the rear housing 112. The lower frame 21 may support, e.g., the collecting device 13. The lower frame 21 may support, e.g., a lower end of the side frame 22. Specifically, the lower end of the side frame 22 may be positioned at two opposite ends of the upper surface of the lower frame 21.

According to an embodiment, the side frame 22 may constitute at least a portion of the side surface of the food waste disposer 1. For example, a pair of side frames 22 may be formed on two opposite side surfaces of the food waste disposer 1. The side frame 22 may be disposed to be perpendicular to, e.g., the lower frame 21. The side frames 22 may be provided to surround, e.g., two opposite sides of the collecting device 13. In other words, at least a portion of the collecting device 13 may be disposed to be surrounded by the housing 112 and the side frame 22.

According to an embodiment, the intermediate frame 23 may be coupled to an upper end of the side frame 22. In other words, the intermediate frame 23 may be supported by the side frame 22. The intermediate frame 23 may be provided, e.g., to partition the placements of the treating device 12 and the collecting device 13 in the food waste disposer 1 or to assist support of the intermediate portion of the food waste disposer 1 to solidify the food waste disposer 1. The intermediate frame 23 may be disposed to be parallel to, e.g., the lower frame 21. The heating device 16 or the treating device 12 may be disposed above the intermediate frame 23.

According to an embodiment, the intermediate frame 23 may include a cut-out portion 231 formed as a portion thereof is cut. The cut-out portion 231 may be formed, e.g., in the front. The cut-out portion 231 may be, e.g., a space through which the transfer pipe 132 for transferring by-products from the treating device 12 to the collecting device 13 passes.

According to an embodiment, a space in which the collecting device 13 is received may be formed by the lower frame 21, the side frame 22, and the intermediate frame 23.

According to an embodiment, the supporting frame 24 may be provided to support the surroundings of the treating device 12 of the food waste disposer 1. For example, a plurality of supporting frames 24 may be disposed at corner portions, respectively, of the intermediate frame 23. The number of supporting frames 24 may be, e.g., four, but is not limited thereto. The treating device 12 may be disposed inside the plurality of supporting frames 24.

According to an embodiment, the accommodating frame 25 may be provided to receive the treating device 12. The accommodating frame 25 may be disposed, e.g., between the upper frame 26 and the intermediate frame 23. The accommodating frame 25 may be disposed, e.g., inside a plurality of supporting frames 24. The accommodating frame 25 may be provided, e.g., in a substantially cylindrical shape with open upper and lower portions.

According to an embodiment, the upper frame 26 may be provided to form an upper surface of the food waste disposer 1 when the cover member 17 is opened. According to an embodiment, the upper frame 26 may include an opening 261, a hinge mounting portion 262, a flow path connecting portion 263, or a flow path opening/closing part 264.

According to an embodiment, the opening 261 may be formed in front of the upper frame 26. The opening 261 may be provided, e.g., to allow the chamber 121 to pass through. Accordingly, a portion of the chamber 121 may be disposed to protrude upward from the upper frame 26. The user may put food waste through the opening 1211 of the chamber 121 protruding upward of the upper frame 26. The shape and size of the opening 261 may correspond to the cross-sectional shape or size of the chamber 121. The opening 261 may be, e.g., circular, but is not limited thereto.

According to an embodiment, the hinge mounting portion 262 may be formed behind the upper frame 26. The hinge 172 of the cover member 17 may be mounted on the hinge mounting portion 262. Accordingly, the cover member 17 may rotate about the hinge 172 with respect to the upper frame 26.

According to an embodiment, the flow path connecting portion 263 may be provided to allow the gas in the chamber 121 to be discharged to the deodorizer 14. The flow path connecting portion 263 may be disposed, e.g., at a position adjacent to the opening 261. The flow path connecting portion 263 may connect, e.g., the flow path of the discharge pipe 178 and the flow path of the exhaust pipe 141 of the deodorizer 14. Accordingly, the gas in the chamber 121 may pass through the discharge pipe 178 and the flow path connecting portion 263 to the exhaust pipe 141 of the deodorizer 14.

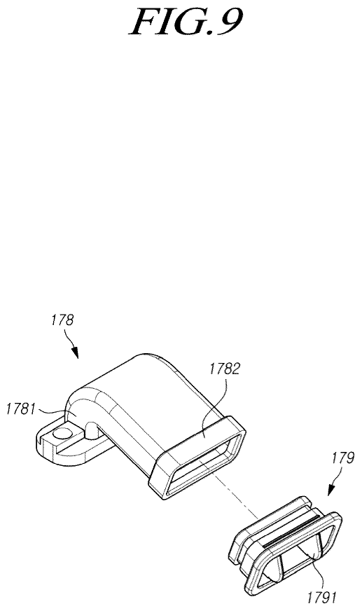

According to an embodiment, the flow path opening/closing part 264 may be provided to open and close the flow path connecting portion 263. The flow path opening/closing part 264 may be provided, e.g., to maintain the flow path connecting portion 263 in a closed state when normal, and to open the flow path connecting portion 263 when one surface is pressed. The flow path opening/closing part 264 may be opened, e.g., by being pressed by a protrusion (e.g., the protrusion 1791 of FIG. 9) of the end cap 179 to be described below.

According to an embodiment, the flow path opening/closing part 264 may include a hinge 2641 or a flow path opening/closing surface 2642. For example, when there is no force applied to the flow path opening/closing surface 2642, i.e., when no external force is applied to the flow path opening/closing surface 2642, the hinge 2641 may be formed so that the flow path opening/closing surface 2642 maintains the closed state of the flow path connecting portion 263. The hinge 2641 may have, e.g., an elastic force in a direction in which the flow path opening/closing surface 2642 closes the flow path connecting portion 263.

According to an embodiment, the flow path opening/closing part 264 may be disposed in the exhaust pipe 141 of the deodorizer 14. For example, a portion of the flow path opening/closing part 264 may be provided to be fixed to the exhaust pipe 141 at a portion where the flow path connecting portion 263 and the exhaust pipe 141 of the deodorizer 14 are connected.

FIG. 8A is a bottom view illustrating a cover member of a food waste disposer according to an embodiment of the disclosure. FIG. 8B is a bottom view with a grill removed from FIG. 8A.

The cover member 17 illustrated in FIGS. 8A and 8B may be identical in whole or part to the cover member 17 illustrated in FIGS. 1 to 7.

According to an embodiment, the grill 176 may include a plurality of holes 1761a. The plurality of holes 1761a may be, e.g., a space through which air flowing by the convective fan 175 passes. The remaining portions of the grill 176 except for the plurality of holes 1761a may be formed to prevent air from penetrating. The grill 176 may be provided to allow air to flow only through the plurality of holes 1761a, thereby preventing food waste finely crushed in the chamber 121 from be flown to the fan 175 by convection. The grill 176 may be, e.g., circular, but is not limited thereto.

According to an embodiment, the grill 176 may include a non-through portion 1762 or a through portion 1761. The grill 176 may have, e.g., the non-through portion 1762 disposed at the central portion thereof, and the through portion 1761 disposed to surround the non-through portion 1762. The non-through portion 1762 may be, e.g., a portion provided so that air does not penetrate. The non-through portion 1762 may have, e.g., a diameter smaller than that of the convective fan 175.

The non-through portion 1762 may be positioned so that, e.g., a center thereof corresponds to a rotation axis of the convective fan 175. The through portion 1761, e.g., may be a portion through which air passes. The through portion 1761 may include, e.g., a plurality of holes 1761a. Accordingly, air may move between the fan 175 and the chamber 121 through the plurality of holes 1761a of the through portion 1761.

The grill 176 may have, e.g., a non-through portion 1762 provided in a central portion thereof, thereby preventing food waste crushed by an upward airflow formed in the center of the chamber 121 from being stuck in the fan 175. Further, the non-through portion 1762 may be provided to bypass, e.g., an upward airflow formed in the center of the chamber 121 to the outside. In other words, as the air rises to the outside of the non-through portion 1762, it is possible to prevent the crushed food waste from getting stuck in the fan 175. Further, as illustrated in FIG. 8B, when the discharge port 178a of the discharge pipe 178 is positioned at an upper side outside of the fan 175, the upward flow of air may be bypassed toward the discharge port 178a to additionally prevent the crushed food waste from getting stuck in the fan 175.

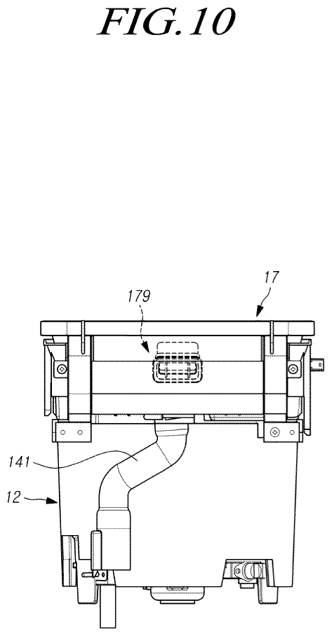

FIG. 9 is a view illustrating a discharge pipe and an end cap of a cover member according to an embodiment of the disclosure. FIG. 10 is a rear view illustrating a cover member of a food waste disposer according to an embodiment of the disclosure.

The discharge pipe 178 and the end cap 179 illustrated in FIGS. 9 and 10 may be identical in whole or part to the discharge pipe 178 and the end cap 179 of the cover member 17 illustrated in FIGS. 1 to 7.

According to an embodiment, the discharge pipe 178 may be detachably coupled to the end cap 179. As another example, the end cap 179 may be integrally formed with the discharge pipe 178. The discharge pipe 178 may be formed so that, e.g., the discharge port 178a of the first end portion 1781 is perpendicular. The discharge pipe 178 may have a curved shape so that the gas in the chamber 121 may pass through the discharge port 178a and move to the flow path connecting portion 263 or the deodorizer 14. In other words, as illustrated, the second end 1782 of the discharge pipe 178 may be disposed to face in an oblique direction.

According to an embodiment, the end cap 179 may be formed to be at least partially hollow therein to allow air to pass. According to an embodiment, the end cap 179 may include a protrusion 1791. The protrusion 1791 may be provided to press, e.g., the flow path opening/closing part 264. When the protrusion 1791 presses the flow path opening/closing part 264, the flow path opening/closing part 264 may be opened. The protrusion 1791 may be formed to protrude, e.g., in a direction opposite to a portion where the end cap 179 is coupled to the discharge pipe 178. In other words, the end cap 179 may be formed to protrude outward when coupled to the discharge pipe 178.

The cross section of the end cap 179 may have a shape corresponding to, e.g., the cross section of the second end 1782 of the discharge pipe 178. The cross section of the end cap 179 may have, e.g., a substantially rectangular shape. The cross section of the end cap 179 may be smaller than, e.g., the cross section of the second end 1782 of the discharge pipe 178. Accordingly, the end cap 179 may be inserted into and coupled to the second end 1782 of the discharge pipe 178, but is not limited thereto.

The discharge pipe 178 may be disposed so that, e.g., the second end 1782 faces the rear side of the food waste disposer 1. In other words, the protrusion 1791 of the end cap 179 coupled to the second end 1782 of the discharge pipe 178 may be disposed to face rearward of the food waste disposer 1. The protrusion 1791 of the end cap 179 may protrude, e.g., toward the hinge 172 of the cover member 17. Corresponding thereto, the deodorizer 14 may be disposed behind the food waste disposer 1.

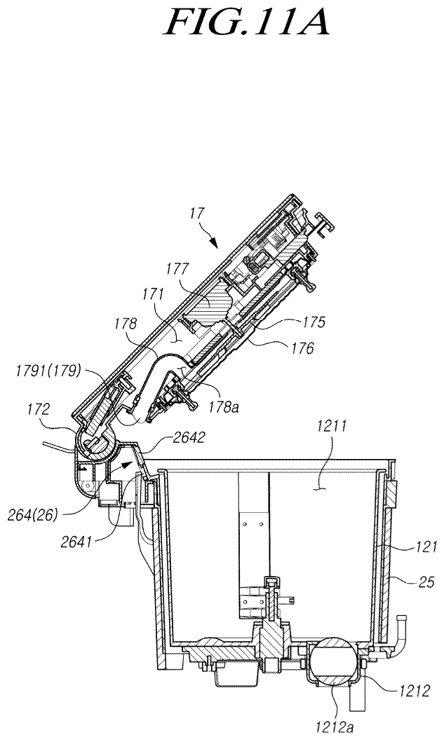

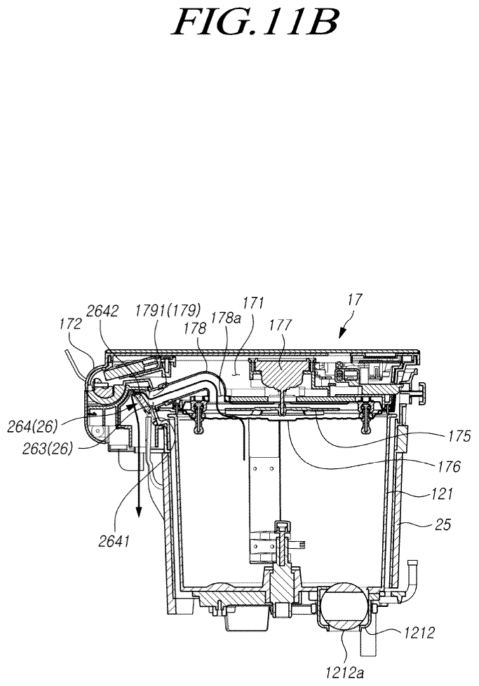

FIG. 11A is a cross-sectional view illustrating a state in which a cover member according to an embodiment of the disclosure is open. FIG. 11B is a cross-sectional view illustrating a state in which a cover member according to an embodiment is closed.

The cover member 17, the upper frame 26, and the chamber 121 illustrated in FIGS. 11A and 11B may be identical in whole or part to the upper frame 26 and the chamber 121 of the cover member 17 illustrated in FIGS. 1 to 7.

As illustrated in FIG. 11A, when the cover member 17 is opened, the flow path opening/closing part 264 may be in a closed state. Since the discharge pipe 178 and the end cap 179 move with the rotation of the cover member 17, the discharge pipe 178 and the end cap 179 may move away from the flow path opening/closing part 264. Accordingly, when the cover member 17 is opened so that the protrusion 1791 of the end cap 179 does not press the flow path opening/closing part 264, the flow path opening/closing part 264 may be closed. In other words, the flow path between the flow path connecting portion 263 and the deodorizer 14 may be blocked.

By closing the flow path opening/closing part 264, foreign substances may be prevented from being introduced into the deodorizer 14 from the outside. For example, when the user opens the cover member 17 and puts the food waste into the chamber 121, a portion of the food waste may be prevented from being introduced into the deodorizer 14. Further, as the flow path opening/closing part 264 closes the flow path connecting portion 263, external dust or other foreign substances may be prevented from flowing into the deodorizer 14 when the cover member 17 is opened. As a result, the service life or performance of the deodorizing part 142 (e.g., the activated carbon filter 143 or the optical filter module 144) in the deodorizer 14 may be enhanced.

As illustrated in FIG. 11B, when the cover member 17 is closed, the flow path opening/closing part 264 may be in an open state. Specifically, if the cover member 17 is closed to cover the upper portion of the chamber 121, the discharge pipe 178 rotating together with the cover member 17 may also be connected to the flow path connecting portion 263. In this case, the second end portion 1782 of the discharge pipe 178 and the flow path connecting portion 263 may be connected to be adjacent to each other. Accordingly, the protrusion 1791 of the end cap 179 positioned at the second end portion 1782 of the discharge pipe 178 may press the flow path opening/closing part 264. The flow path opening/closing part 264 may be opened by being pressed by the protrusion 1791 of the end cap 179. The gas in the chamber 121 may move to the deodorizer 14 through the discharge pipe 178, the end cap 179, and the flow path connecting portion 263. As a result, the flow path connecting portion 263 may be opened only when the cover member 17 is closed.

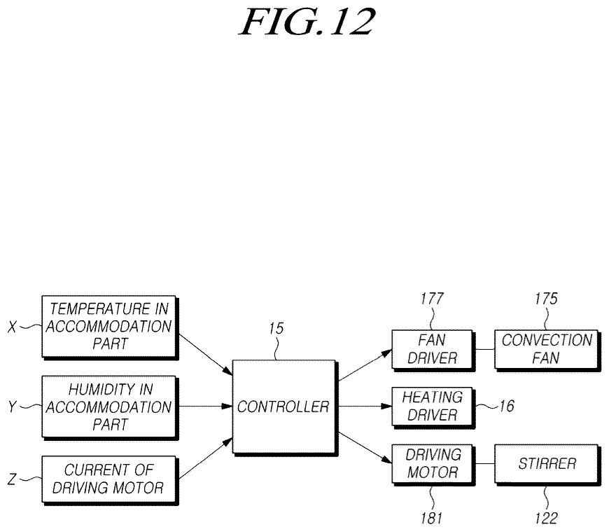

FIG. 12 is a block view illustrating a food waste disposer according to an embodiment of the disclosure.

As the components illustrated in FIG. 12, the components operated by the controller among the components of the food waste disposer 1 of FIGS. 1 to 7 are mainly illustrated, but this is exemplary, and components other than those illustrated may also be controlled by the controller 15. FIG. 12 is a block diagram illustrating a method for controlling the operation of devices used when crushing, stirring, or heating food waste using information about torque of the driving motor 181 or the temperature or moisture, by the controller 15.

Referring to FIG. 12, a food waste disposer 1 according to an embodiment of the disclosure may include a housing 11, a treating device 12, a collecting device 13, a deodorizer 14, a heating device 16, a cover member 17, or a controller 15. The controller 15, the fan driver 177, the convective fan 175, the heating device 16, the driving device 18, or the stirrer 122 illustrated in FIGS. 1 to 7 may be identical in whole or part to the controller 15, the fan driver 177, the convective fan 175, the heating device 16, the driving device 18, or the stirrer 122 illustrated in FIGS. 1 to 7.

For example, the controller 15 may receive information about the temperature X inside the chamber from the temperature detector 127, and receive information about the humidity Y inside the chamber from the humidity detector 128. Further, the controller 15 may measure the current Z flowing through the driving motor 181 driven to rotate the stirrer 122, or may receive the current Z from an device that detects an external current. For example, the controller 15 may estimate the torque of the driving motor 181 using the current Z flowing through the driving motor 181.

After the food waste is introduced into the chamber 121, the controller 15 may start treating the food waste automatically or by a user input. The controller 15 may operate the heating device 16 and the driving device 18 to treat food waste.

The heating device 16 may be provided, e.g., outside the chamber 121. The heating device 16 may be provided adjacent to, e.g., the lower surface of the chamber 121. The heating device 16 may increase, e.g., the temperature X in the chamber 121 to dry food waste.

The driving device 18 may be provided to rotate, e.g., the stirrer 122 disposed in the chamber 121. In other words, as the driving device 18 is operated, rotational power may be transferred to the stirrer 122 to crush food waste, and the crushed food waste may be stirred.

While the food waste treatment is performed, the controller 15 may continuously monitor the temperature X, the humidity Y, or the current Z flowing through the driving motor 181 in the chamber 121. For example, the controller 15 may receive information about the temperature X from a temperature detector (e.g., the temperature detector 127 of FIG. 4) disposed to detect the temperature X in the chamber 121. For example, the controller 15 may receive information about the humidity Y from a humidity detector (e.g., the humidity detector 128 of FIG. 4) disposed to detect the humidity Y in the chamber (e.g., the chamber 121 of FIG. 4).

The controller 15 may drive the fan driver 177 when food waste treating is performed and a predetermined condition is met. The fan driver 177 may be provided, e.g., in an inner space (e.g., the inner space 171 of FIG. 4) of a cover member (e.g., the cover member 17 of FIG. 4). The cover member (e.g., the cover member 17 of FIG. 4) may be provided, e.g., above the chamber (e.g., the chamber 121 of FIG. 4).

For example, when the temperature in the chamber 121 is equal to or larger than the predetermined temperature value, the controller 15 may perform control to operate the fan driver 177. When the fan driver 177 operates, rotational power may be transferred to the convective fan 175 to cause convection in the chamber 121.

The controller 15 may adjust the operating intensity of the fan driver 177 using the humidity Y in the chamber 121. For example, the controller 15 may increase the operating intensity of the fan driver 177 as the humidity in the chamber (e.g., the chamber 121 of FIG. 4) increases.

The controller 15 may determine the end time of the food waste treatment. For example, the controller 15 may determine whether the grinding and drying of the food waste in the chamber 121 is completed using the humidity Y in the chamber (e.g., the chamber 121 of FIG. 4) or the current Z flowing through the driving motor 181. For example, when the humidity Y in the chamber (the chamber 121 of FIG. 4) is less than a predetermined humidity value and the current Z flowing through the driving motor 181 is less than a predetermined current value, the controller 15 may determine that the grinding and drying of the food waste is completed, but is not limited thereto. The controller 15 may set a food waste treating time in advance and end the food waste treating after the predetermined time.

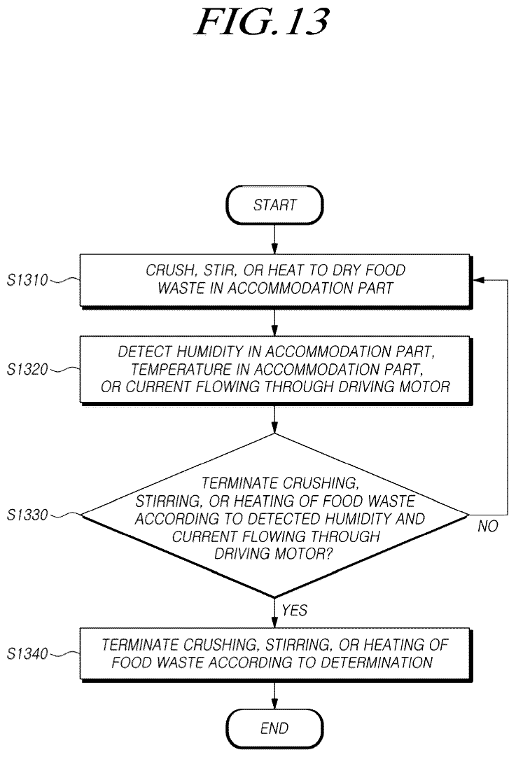

FIG. 13 is a flowchart illustrating a method for controlling a food waste disposer according to an embodiment of the disclosure.

FIG. 13 is a flowchart illustrating an overall process for drying and treating food waste in the chamber 121 by the controller 15. Hereinafter, it is described with reference to FIGS. 4, 12, and 13.

The controller 15 may perform control for drying the food waste by grinding, stirring, or heating the food waste in the chamber 121 (S1310). The controller 15 may control the stirrer 122 to crush or stir food waste by operating, e.g., the driving motor 181. The controller 15 may dry food waste by operating, e.g., the heating device 16. Water vapor generated by heating may be moved to the deodorizer 14 through the discharge pipe 178. In this case, the controller 15 may operate the fan driver 177 to cause convection in the chamber 121 to enhance the drying speed.

The controller 15 may monitor the humidity Y in the chamber 121, the temperature X in the chamber 121, or the current Z flowing through the driving motor 181 (S1320). For example, the controller 15 may continuously monitor the humidity Y in the chamber 121, the temperature X in the chamber 121, or the current Z flowing through the driving motor 181 while performing operation S1310. The humidity Y may be detected by, e.g., the humidity detector 128. The temperature X may be detected by, e.g., the temperature detector 127. The controller 15 may receive, e.g., information about the temperature X or humidity Y from the temperature detector 127 or the humidity detector 128. For example, the controller 15 may directly measure the current Z flowing through the driving motor 181, but is not limited thereto, and may be transferred from a separate sensor that detects a current.

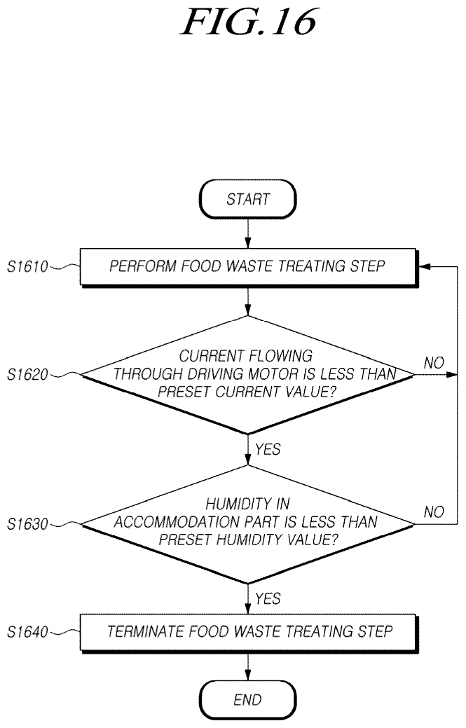

The controller 15 may determine whether to end grinding, stirring, or heating of the food waste according to the detected humidity Y and the current Z flowing through the driving motor 181 (S1330). The controller 15 may estimate, e.g., the degree of drying of the food waste using the humidity Y in the chamber 121. The controller 15 may estimate, e.g., the degree of grinding of food waste using the current Z of the driving motor 181. Details are described below in connection with FIG. 16.

The controller 15 may end grinding, stirring, or heating of the food waste as determined in operation S1330 (S1440). For example, the controller 15 may stop the operation of the fan driver 177, the heating device 16, or the driving motor 181 when it is determined that the grinding, stirring, or heating of the food waste should be terminated in operation S1430.

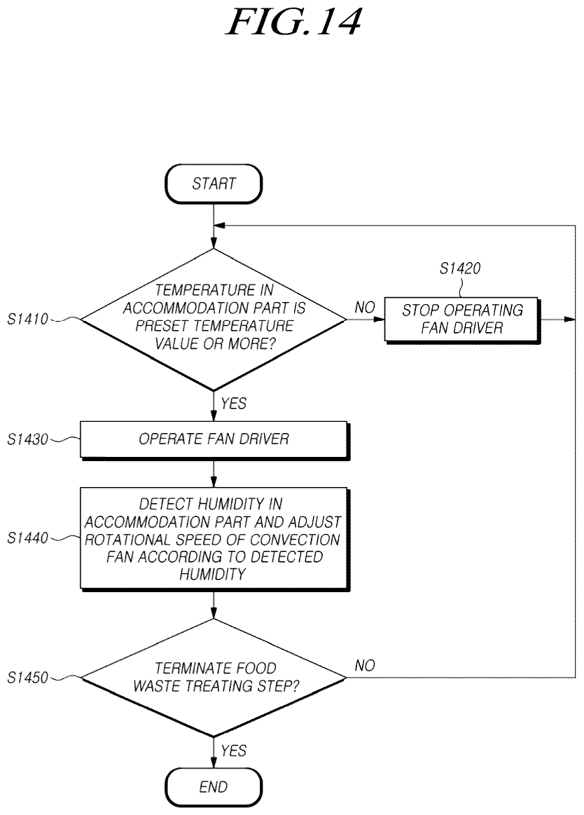

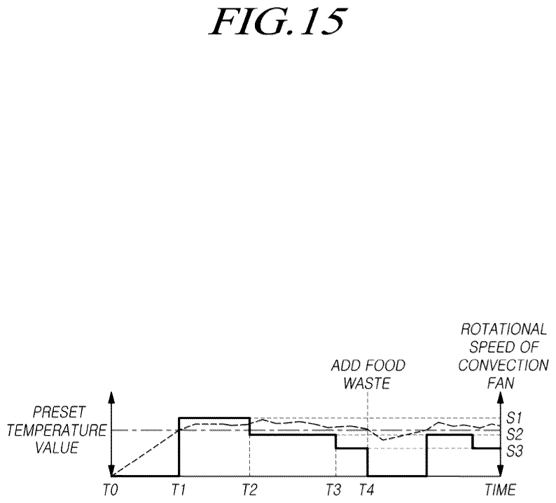

FIG. 14 is a flowchart illustrating a method for controlling an operation of a fan driver for rotating a convective fan in operation S1310 of FIG. 13. FIG. 15 is a graph illustrating an operation of a fan driver during an operation of a food waste disposer according to an embodiment of the disclosure.

Hereinafter, it is described with reference to FIGS. 4, 12, 14, and 15.

The controller 15 may determine whether the temperature X in the chamber is larger than or equal to a predetermined temperature value (S1410). The controller 15 may periodically detect the temperature X in the chamber 121 every preset time interval. Whenever the controller 15 detects the temperature X, the controller 15 may determine whether the temperature X in the chamber 121 is larger than or equal to the predetermined temperature value. The controller 15 may determine whether the average value is larger than or equal to the predetermined temperature value using, e.g., the average value of the temperature X in the chamber 121 for a predetermined time.

When the controller 15 does not determine that the temperature X in the chamber 121 is larger than or equal to the predetermined temperature value, the controller 15 may stop the operation of the fan driver 177 (S1420). When the fan driver 177 is already stopped, the controller 15 may maintain the fan driver 177 in the stopped state. The controller 15 may perform operation S1410 after stopping the operation of the fan driver 177.

When the controller 15 determines that the temperature X in the chamber 121 is equal to or larger than the predetermined temperature value, the controller 15 may perform control to operate the fan driver 177 (S1430). When the fan driver 177 is operated when the temperature X in the chamber 121 is low, a time for raising the temperature X in the chamber 121 may be delayed. Accordingly, the controller 15 may shorten the drying time of the food waste in the chamber 121 by operating the fan driver 177 only when it is determined that the temperature X in the chamber 121 is larger than or equal to the predetermined temperature value.

Referring to the graph of FIG. 15, the fan driver 177 is not driven while the temperature X in the chamber 121 increases in the section between T0 and T1. The controller 15 may drive the fan driver 177 at time T1 when the temperature X in the chamber 121 reaches the predetermined temperature value.

When the fan driver 177 is operated, the controller 15 may detect the humidity Y in the chamber 121 and adjust the rotational speed of the convective fan 175 according to the detected humidity (S1440). For example, the controller 15 may perform control to increase the rotational speed of the convective fan 175 as the humidity Y in the chamber 121 increases. When the humidity Y in the chamber 121 is high, the fan 175 may be quickly rotated to shorten the drying time. Conversely, when the humidity Y in the chamber 121 is low, it is sufficient to cause only convection in the chamber 121, and thus power may be reduced by lowering the rotational speed of the fan 175.

Referring to the graph of FIG. 15, the controller 15 may control the fan driver 177 to vary the rotational speed according to the humidity Y in the chamber 121 in the section T1 to T4 driven by the fan driver 177. For example, as illustrated in FIG. 15, the controller 15 may adjust the rotational speed of the fan driver 177 in three stages, but is not limited thereto. The controller 15 may adjust the rotational speed of the fan driver 177 by dividing the fan driver 177 into fewer or more stages. Hereinafter, the rotational speeds are referred to as a first rotational speed S1, a second rotational speed S2, or a third rotational speed S3, respectively, in the order from the highest speed. For example, when the humidity Y in the chamber 121 is larger than or equal to the first humidity value, the controller 15 may drive the fan driver 177 at the first rotational speed S1. For example, when the humidity Y in the chamber 121 is less than the first humidity value and is larger than or equal to the second humidity value less than the first humidity value, the controller 15 may drive the fan driver 177 at the second rotational speed S2. For example, when the humidity Y in the chamber 121 is less than the second humidity value, the controller 15 may drive the fan driver 177 at the third rotational speed S3.

At time T1 when the fan driver 177 starts to operate, water vapor may be generated in the chamber 121 by heating to form a high humidity Y (e.g., humidity larger than or equal to the first humidity value). Accordingly, the controller 15 may drive the fan driver 177 at the first rotational speed S1 between time T1, which is the initial time when the fan driver 177 is driven, and time T2. After time T2, when the humidity Y in the chamber 121 is less than the first humidity value and larger than or equal to the second humidity value, the controller 15 may drive the fan driver 177 at the second rotational speed S2. After time T3, when the humidity Y in the chamber 121 is less than the second humidity value, the controller 15 may drive the fan driver 177 at the third rotational speed S3. As such, the controller 15 may reduce the rotational speed of the fan driver 177 over time, thereby reducing the power consumed by the food waste disposer 1.

In the graph of FIG. 15, the controller 15 divides the range of the humidity Y to stepwise control the rotational speeds of the fan driver 177. However, the disclosure is not limited thereto, the controller 15 may form a linear relationship between the humidity Y and the rotational speed of the fan driver 177 to change the rotational speed of the fan driver 177 in real time according to the detected humidity Y. In other words, as illustrated in the graph of FIG. 15, the rotational speed of the fan driver 177 may not decrease stepwise rather than decreasing linearly.

Additionally, when the user opens the cover member 17 and puts new food waste after time T4, the temperature X and humidity Y in the chamber 121 may change rapidly. The controller 15 may periodically perform operation S1410 to control the operation of the fan driver 177 in real time according to a change in the temperature X. When the temperature X in the chamber 121 is less than the predetermined temperature value as a result of inputting the food waste, the controller 15 may stop the operation of the fan driver 177 by performing operation S1420.

The controller 15 may determine whether the food waste treating operation is finished (S1450). Here, the food waste treating operation may refer to a operation of grinding, stirring, or heating the food waste to dry the food waste. When the controller 15 determines that the food waste treating operation ends, the controller 15 may end the operation of the fan driver 177. When the controller 15 does not determine that the food waste treating operation ends, the controller 15 may perform operation S1410. A method for determining whether the food waste treating operation ends is described in detail with reference to FIG. 16.

FIG. 16 is a flowchart illustrating a method for determining an end time of a food waste treating operation by a food waste disposer according to an embodiment of the disclosure.

The controller 15 may monitor the humidity Y in the chamber 121 or the current Z of the driving motor 181 in real time every preset time interval while performing the food waste treating operation. Operation S1330 of FIG. 13 is described in detail with reference to FIG. 16. Hereinafter, it is described with reference to FIGS. 4, 12, and 16.

The controller 15 may perform a food waste treating operation (S1610). The food waste treating operation may refer to an operation in which the controller 15 crushes, stirs, or heats to dry food waste in the chamber 121 by controlling the fan driver 177, the heating device 16, or the driving motor 181 as illustrated in FIG. 12.

The controller 15 may monitor the current Z flowing in the driving motor 181 or the humidity Y in the chamber 121 periodically every preset time interval while performing the food waste treating operation.

In the food waste treating operation S1610, the food waste in the chamber 121 needs to be completely crushed and dried. Since each type of food waste has different elements such as the amount and intensity of moisture, the time required to treat the food waste may vary for each food waste. Further, the time required in the food waste treating operation S1610 may vary depending on the amount of food waste put into the chamber 121. In the method for controlling the food waste disposer according to an embodiment of the disclosure, the time when the food waste treatment is finished may be identified in real time using the current Z flowing through the driving motor 181 and the humidity Y in the chamber 121.

Here, operation S1610 may correspond to operation S1310 of FIG. 13.

The controller 15 may determine whether the current Z flowing through the driving motor 181 is less than a preset current value (S1620). The controller 15 may estimate the torque of the driving motor 181 using the current Z flowing through the driving motor 181. The preset current value may vary according to the specification of the driving motor 181. The preset current value may be, e.g., 0.5 A. The controller 15 may set, e.g., a first measurement time. The controller 15 may calculate an average value of the current Z flowing through the driving motor 181 during the first measurement time and compare its magnitude with a predetermined current value. As another example, the controller 15 may determine that the current Z flowing through the driving motor 181 is less than the predetermined current value when the number of times when the current Z is detected within the first measurement time is less than the predetermined current value is a predetermined number or more. The above-described method is an exemplary method for reducing the influence of noise generated when measuring the current Z, and various methods for estimating the torque of the driving motor 181 using the current Z flowing through the driving motor 181 may be used within the technical scope of those skilled in the art. When the controller 15 does not determine that the current Z flowing through the driving motor 181 is less than a preset current value, the controller 15 may perform operation S1610.

When it is determined that the current Z flowing through the driving motor 181 of the controller 15 is less than the predetermined current value, it may be determined whether the humidity Y in the chamber 121 is less than the predetermined humidity value (S1630). The controller 15 may estimate the drying progress of the food waste using the humidity Y in the chamber 121. For example, when the humidity Y in the chamber 121 is less than the predetermined current value, the controller 15 may determine that the drying process in the chamber 121 ends. The preset humidity value may be, e.g., 17%, but is not limited thereto. The controller 15 may set a second measurement time. For example, the controller 15 may determine whether the average value of the humidity Y measured during the second measurement time is less than a preset humidity value. As another example, the controller 15 may determine whether all of the humidity Y measured during the second measurement time is less than a predetermined humidity value. The above-described method is an exemplary method for reducing the influence of noise generated while the humidity detector 128 detects the humidity Y, and various methods for measuring the humidity Y while reducing the influence of noise of a sensor within the technical scope of those skilled in the art may be used. When the controller 15 does not determine that the humidity Y in the chamber 121 is less than the predetermined humidity value, the controller 15 may perform operation S1610.

Here, the controller 15 may perform operation S1620 and operation S1630 in a different order. Operations S1620 and S1630 may be included in operation S1330 of FIG. 13.

When the controller 15 determines that the humidity Y in the chamber 121 is less than the predetermined humidity value, the controller 15 may end the food waste treating operation (S1640). For example, the controller 15 may stop the operation of the fan driver 177, the heating device 16, or the driving motor 181 to terminate the food waste treating operation. Here, operation S1640 may correspond to operation S1340 of FIG. 13.

The terms as used herein are provided merely to describe some embodiments thereof, but are not intended to limit the disclosure. As used herein, the singular forms “a,” “an,” and “the” are intended to include the plural forms as well, unless the context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, the term ‘and/or’ should be understood as encompassing any and all possible combinations by one or more of the enumerated items. As used herein, the terms “include,” “have,” and “comprise” are used merely to designate the presence of the feature, component, part, or a combination thereof described herein, but use of the term does not exclude the likelihood of presence or adding one or more other features, components, parts, or combinations thereof. As used herein, the terms “first” and “second” may modify various components regardless of importance and/or order and are used to distinguish a component from another without limiting the components.

As used herein, the terms “configured to” may be interchangeably used with the terms “suitable for,” “having the capacity to,” “designed to,” “adapted to,” “made to,” or “capable of” depending on circumstances. The term “configured to” does not essentially mean “specifically designed in hardware to.” Rather, the term “configured to” may mean that a device can perform an operation together with another device or parts. For example, a ‘device configured (or set) to perform A, B, and C’ may be a dedicated device to perform the corresponding operation or may mean a general-purpose device capable of various operations including the corresponding operation.

Meanwhile, the terms “upper side”, “lower side”, and “front and rear directions” used in the disclosure are defined with respect to the drawings, and the shape and position of each component are not limited by these terms.

In the disclosure, the above-described description has been made mainly of specific embodiments, but the disclosure is not limited to such specific embodiments, but should rather be appreciated as covering all various modifications, equivalents, and/or substitutes of various embodiments.

What is claimed is:

1. A food waste disposer, comprising:
a chamber to receive food waste to be treated;
a deodorizer to deodorize gas and discharge the deodorized gas to an outside of the chamber; and
a cover member arrangeable to cover the chamber, wherein the cover member includes:
a fan arrangeable to face inward of the chamber and configured to generate convection in the chamber while being driven; and
a discharge pipe including a discharge port formed at a first end portion arrangeable outside the fan, and a second end portion opposite to the first end portion that is connectable to the deodorizer.

2. The food waste disposer of claim 1, further comprising:
a flow path opening/closing part to open and close a flow path between the discharge pipe and the deodorizer; and
an end cap, arrangeable at the second end portion of the discharge pipe, and including a protrusion to press the flow path opening/closing part.

3. The food waste disposer of claim 2, wherein while the cover member closes the chamber, the protrusion is configured to press the flow path opening/closing part to open the flow path between the discharge pipe and the deodorizer, and/or while the cover member opens the chamber, the protrusion is configured to close the flow path between the discharge pipe and the deodorizer by not pressing the flow path opening/closing part.

4. The food waste disposer of claim 2, wherein the protrusion is formed to protrude from the end cap in a direction in which the gas in the chamber is discharged.

5. The food waste disposer of claim 2, further comprising an upper frame including a flow path connecting portion under the cover member to support the cover member and connect the flow path of the discharge pipe and the flow path of the deodorizer,
wherein the flow path opening/closing part is partially fixed to the upper frame, and is configured to open and close the flow path connecting portion.

6. The food waste disposer of claim 1, wherein the fan is a fan and the discharge port is positioned above the fan.

7. A food waste disposer to perform a process of stirring, grinding, or heating food waste, the food waste disposer comprising:
a treating device including a chamber to accommodate food waste, a stirrer arrangeable in the chamber to stir or crush the food waste, and a driving device to rotate the stirrer;
a heating device arrangeable outside the chamber;
a cover member to cover the chamber, the cover member including a fan arrangeable to face inward of the chamber and a fan driver to rotate the fan; and
a controller to control an overall operation of the driving device, the heating device, or the fan driver,
wherein the controller determines an end time of a treating process based on humidity in the chamber and current flowing through the driving device.

8. The food waste disposer of claim 7, wherein the controller controls an operation of the driving device, the heating device, or the fan driver using a temperature in the chamber, a humidity in the chamber, or a current value flowing through the driving device.

9. The food waste disposer of claim 7, wherein the controller controls to terminate the treating process when the humidity in the chamber is a preset humidity value or less, and the current flowing through the driving device is a preset current value or less.

10. The food waste disposer of claim 7, wherein the controller controls to increase a rotational speed of the fan as the humidity in the chamber increases.

11. The food waste disposer of claim 7, wherein the controller controls to operate the fan driver based on a temperature in the chamber being a preset temperature value or more.

12. A method of controlling a food waste disposer, the method comprising:
treating food waste in a chamber by stirring, crushing, or heating to dry;
detecting a humidity in the chamber or a magnitude of a current flowing through a driving motor driven to stir the food waste in the chamber; and
determining whether to terminate treating the food waste according to the detected humidity and the magnitude of the current flowing through the driving motor.

13. The method of claim 12, wherein the treating is terminated based on the humidity in the chamber being less than a predetermined humidity value and the current flowing through the driving motor being less than a predetermined current value in the terminating.

14. The method of claim 12, wherein in the treating, based on a temperature in the chamber being a predetermined temperature value or more, a fan driver rotating a fan to convect air in the chamber is operated.

15. The method of claim 12, wherein in the treating, as the humidity in the chamber increases, a rotational speed of a fan convecting air in the chamber increases.
